(19) United States
(12) Patent Application Publication
(10) Pub. No.: US 2004/0166574 A1
(43) Pub. Date: Aug. 26, 2004

(54) Microorganism

(76) Inventor: John I. Antonius, Fairview, OR (US)

Correspondence Address: GRAYBEAL, JACKSON, HALEY LLP, 155 - 108TH AVENUE NE, SUITE 350, BELLEVUE, WA, 98004-5901, US

(21) Appl. No.: 10/374,455

(22) Filed: Feb. 25, 2003

(51) Int. Cl. C12Q 1/04; C12N 1/12; C12N 1/20

(52) U.S. Cl. 435/252.1; 435/34

(57) ABSTRACT

A novel microorganism identified as Microorganism John Antonius, or MJA, and kits, methods and the like related thereto.

### BACKGROUND

Diseases of the human gastrointestinal system cause untold misery to millions of people, as well as massive expense to society at large. For example, according to the National Digestive Diseases Information Clearinghouse (NIDDK) of the National Institutes of Health (NIH), “digestive diseases cost nearly $107 billion in direct health care expenditures in 1992 . . . [including] diarrheal infections ($4.7 billion); gallbladder disease ($4.5 billion); colorectal cancer ($4.5 billion); liver disease ($3.2 billion); and peptic ulcer disease ($2.5 billion).” http://www.niddk.nih.gov/health/digest/pubs/overview/overview. htm#5. The market for sales of anti-ulcerants, alone, in the 12 month period ending March 2002 was $17.8 billion with an annual growth rate of 12%. IMS HEALTH, Drug Monitor, http://www.imshealth.com/public/structure/dispcontent/1,2779,1039-1039-144111,00.html.

The search for and discovery of microorganisms that might be related to such diseases has been the subject of great interest. For example, a search of U.S. Patent Office records on Jan. 15, 2003 for “pylori” (for the microorganism Helicobactor pylori, which was discovered in 1982 and may be related to ulcers, gastritis and possibly some forms of gastric cancer) found that “pylori” was mentioned in 1327 issued patents, and recited in the claims of 293 patents.

Thus, there has gone unmet a need for the identification of microorganisms that are found in or associated with diseased human gastrointestinal systems. The present systems and methods provide this and other advantages.

### SUMMARY

The present invention comprises a microorganism that is found in humans that have gastrointestinal diseases such as colitis. The microorganism has also been found in humans that have other conditions. This microorganism is referred to herein as Microorganism John Antonius, or MJA, after its discoverer. Such name of the microorganism interchangeable with, and therefore includes, other specific names for the microorganism that may arise, i.e., other specific identifiers of equivalent import used to identify the microorganism. It appears that MJA may cause or be an exacerbating factor in at least one of these diseases. Accordingly, the discovery of MJA is a significant advance in the research about such diseases, and may be a significant advance in the diagnosis or treatment of the diseases.

In one aspect, the present invention comprises substantially purified MJA. In some embodiments, the MJA (ATCC as Accession No. ______) can be isolated and specifically identified. The present invention also includes kits comprising a vessel containing isolated or substantially purified MJA and a label specifically identifying the MJA. The vessel can be a vial, microscope slide or other desired container, and the MJA can be in a cellular or a cellular culture medium, a tissue sample or other desired growth, maintenance or holding substrate.

The present invention also comprises methods of obtaining MJA comprising: a) identifying an animal suspected of being infected with MJA; and, b) withdrawing a sample of tissue from the animal wherein the sample contains MJA. The methods can further comprise specifically identifying MJA from the tissue, substantially purifying the MJA from the tissue and, if desired, growing the MJA in a culture medium or other growth substrate.

Other methods provide for specifically identifying MJA. Such methods can comprise a) providing a sample of tissue suspected of containing MJA; b) examining the sample to determine the presence of MJA; and, c) specifically identifying MJA. Such examining of the sample can include comparing the MJA to the sample of MJA deposited with the ATCC as Accession No. ______ or referring to one or more of the features discussed herein. Such methods can further comprise, prior to the identifying, staining the sample with a stain suitable for MJA, and the examining can comprise magnifying the MJA and creating an image of the MJA. Such magnifying can comprise, for example, visible light microscopy, fluorescent light microscopy or electron microscopy, and the image can comprise a photomicrograph such as an electronphotomicrograph, a visible light photomicrograph or a fluorescence photomicrograph.

These and other aspects, features and embodiments are set forth within this application, including the following Detailed Description and attached drawings. In addition, various references are set forth herein that discuss in more detail certain systems, apparatus, methods and other information; all such references are incorporated herein by reference in their entirety and for all their teachings and disclosures, regardless of where the references may appear in this application. Unless expressly stated otherwise or clear from the context, all embodiments, aspects, features, etc., can be mixed and matched, combined and permuted in any desired manner.

### BRIEF DESCRIPTION OF THE DRAWINGS

FIG. 1 is an electron micrograph of an MJA in a red blood cell (RBC).

FIG. 2 is a 1,000× photomicrograph of MJA in a 1% crystal-violet stained, wet-mounted blood sample from a human with colitis.

FIG. 3 is a 1,000× photomicrograph of MJA in a 1% crystal-violet stained, wet-mounted blood sample from a human with bronchitis and diarrhea.

FIG. 4 is a 1,000× photomicrograph of MJA in a Genta stained colon sample from a human with colitis and polyps of the colon.

FIG. 5 is a 400× photomicrograph of MJA in an H&E stained colon sample from a human with non-specific colitis.

FIG. 6 is a 1,000× photomicrograph of MJA in a Steiner stained colon sample from a human with colitis.

FIG. 7 is a 1,000× photomicrograph of MJA in a Steiner stained vascular muscle of the colon from a human with colitis.

FIG. 8 is a 1,000× photomicrograph of MJA in a preparation stained for electron microscopy. The preparation is a colon sample from a human with colitis.

FIG. 9 is a 400× photomicrograph of MJA in a Steiner stained colon sample from a human with non-specific colitis.

FIG. 10 is a 1,000× photomicrograph of MJA in an H&E stained duodenum sample from a human with abdominal discomfort, fatigue and a general feeling of sickness.

FIG. 11 is a 1,000× photomicrograph of MJA in a Steiner stained polyp from the colon from a human with colitis.

FIG. 12 is a 40× photomicrograph of MJA in a Genta stained colon polyp sample from a human with colitis.

FIG. 13 is a 1,000× photomicrograph of MJA in a Steiner stained male breast sample from a human with gynecomastia.

FIG. 14 is a 1,000× photomicrograph of MJA in an H&E stained salivary gland sample from a human.

FIG. 15 is a 1,000× photomicrograph of MJA in an H&E stained choroid plexus sample from a human that had died from diabetes mellitus.

FIG. 16 is a 1,000× photomicrograph of MJA in a Steiner stained section of yolk sac from a fertilized chicken egg that had been infected with MJA.

### DETAILED DESCRIPTION

Illnesses such as colitis and inflammatory bowel disease (IBD) cause much human suffering. The search for the cause(s) of, and other factors in, such diseases is ongoing. A newly discovered microorganism, MJA, has been found in humans that have these, and other, conditions. MJA may exacerbate or cause one or more of such diseases or conditions. Thus, the discovery of MJA is a significant advance in research about such diseases, and may also be a significant advance in the diagnosis or treatment of the diseases or conditions.

Definitions.

All terms used herein, including those specifically discussed in this section, are used in accordance with their ordinary meanings unless the context or definition clearly indicates otherwise. Also unless indicated otherwise, except within the claims, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

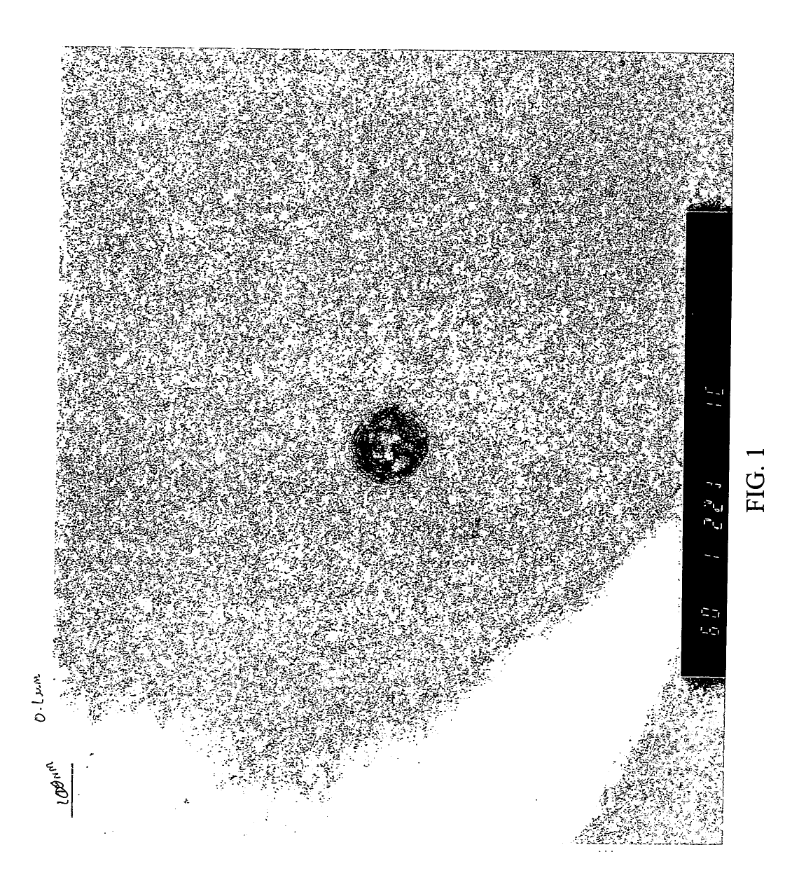

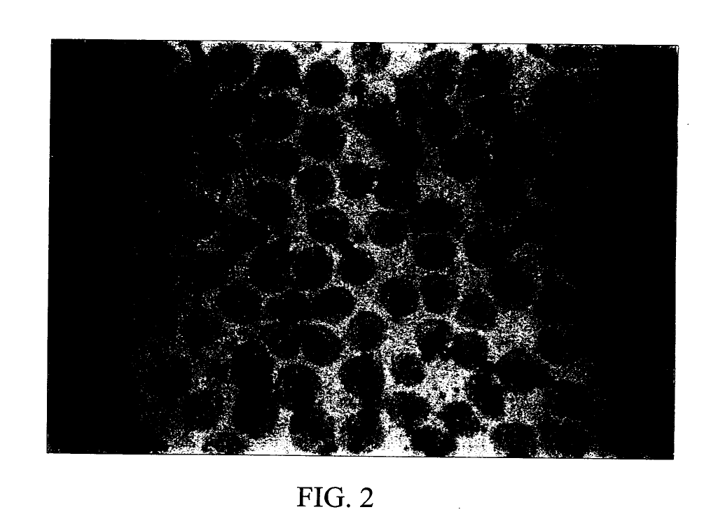

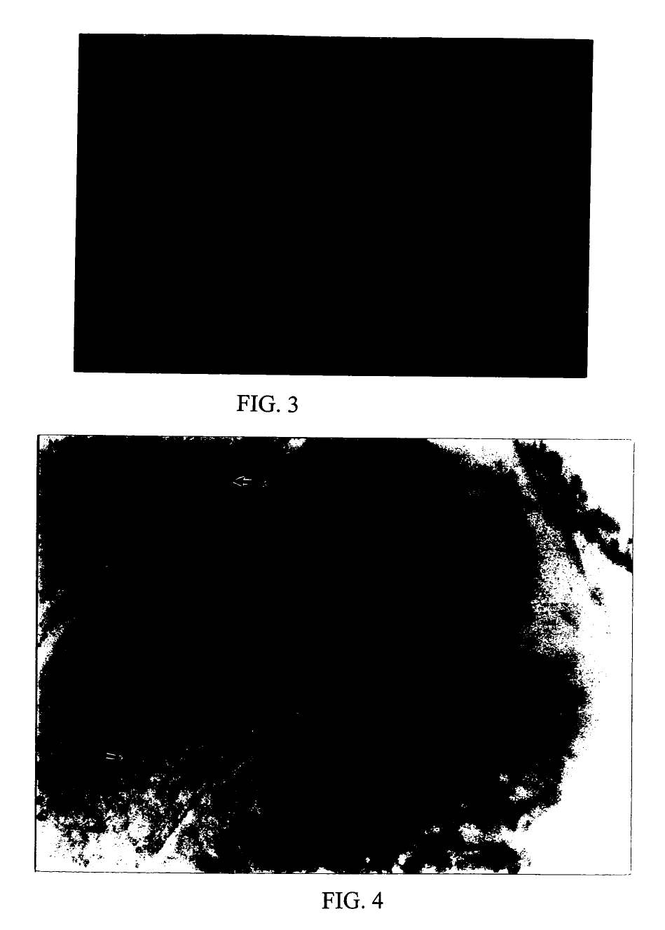

“Microorganism John Antonius (MJA)” is a specific identifier for the microorganism that is the subject of this application and is interchangeable with, and therefore includes, other specific names for the microorganism that may arise, i.e., other specific identifiers of equivalent import used to identify the microorganism, and also includes mutants, recombinants and variants of MJA. Examples of MJA are shown in the electron micrograph of FIG. 1 and in the photomicrographs of FIGS. 2-16. MJA was deposited in February, 2003, with the American Type Culture Collection (ATCC), 10801 University Boulevard, Manassas, Va., U.S.A. 20110-2209, under the provisions of the Budapest Treaty for the International Recognition of the Deposit of Microorganisms for the Purposes of Patent Procedure and was accorded ATCC Accession No. ______. The deposit is provided as a convenience to interested parties, and is not an admission that a deposit is required. A license may be required to make, use, or sell the deposited materials; no such license is granted hereby.

“Isolated” generally means that the material is removed from its original environment (e.g., its natural environment if it is naturally occurring). When referring to MJA, isolated means that the MJA has been separated from its host, for example in a tissue sample, a pure tissue culture, etc. Thus, a naturally-occurring MJA in a living animal is not isolated. But the same MJA, separated by artificial methods from the animal is isolated.

“Substantially purified” refers to MJA removed from its natural environment and separated from a significant portion of the other components of such natural environment. Highly purified indicates that substantially all other components of its natural environment have been removed, for example in an acellular culture medium or in a tissue culture where the tissue is not from the original host of the particular MJA (such as a commercially available tissue or cellular culture medium). Purity can be assayed by standard methods using traditional techniques that are readily apparent to a skilled person in view of the present application.

The scope of the present discussion includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

The discovery of MJA.

While reviewing biopsies from two patients while working in Saudi Arabia, the inventor (a pathologist) saw unidentifiable microorganisms in the tissues. He could see the microorganisms in electron microscopy and micrographs, but could not find them using ordinary investigative procedures such as growing them in culture or using sero-immunological studies using known antibodies against known organisms.

Later, in another laboratory, the inventor reviewed stomach and gastrointestinal tract biopsies of a number of gastrointestinal patients that he could not classify into a specific disease class such as Crohn's disease or ulcerative colitis. In the gastrointestinal tissues of these cases he discovered a microorganism in the smooth muscle layers in the blood vessels and in the epithelium. He also discovered that the blood vessels were blocked by the unidentified microorganism or something associated with the unidentified microorganism. The blocking material when stained with eosin Y was highly fluorescent under UV light but was not visible under white light nor UV light without the eosin Y staining. Such microorganism appeared to be the same as the unidentified microorganism he had seen previously. The blockages indicated that the microorganism may have access to the bloodstream and thus could possibly infect a variety of organs.

The inventor then launched a study of the unidentified microorganism upon returning to the United States. Among other things, he used atypical stains, fluorescent light and regular and electron microscopes. Using the fluorescence, the inventor observed that blood vessels in the colon of patients (often patients with only mild symptoms) were damaged and blocked to a marked degree. This was important, in part, because most pathologists believed the ulcerations in the colon were like that of other ulcerating diseases, such as typhoid, dysentery and cholera, where the organisms were on the surface. However, the inventor did not find MJA at that time on the surface, or lumen, of the colon, but rather deep in the tissue (MJA is typically difficult to find on the lumen).

Samples from two cases were examined by growing the microorganisms in chick embryo tissue culture. Biopsies from the cases were taken at the time of surgery, the biopsies were placed in sterile physiological saline with 50 μg/ml for approximately 2 hours, then rinsed several times in saline. The biopsies were then ground up in 1 ml of physiological saline using a mortar and pestle, the resulting suspension was aspirated into a syringe then 0.2 ml was injected into the yolk sac of 6 day old chick embryos. They were incubated 6 days then harvested. Tissue from the yolk sac membrane was placed in 10% formalin and histologic sections were made and Steiner stained.

The presence of unidentified microorganisms was confirmed, which microorganisms were located in the embryo cells in locations that substantially correlated to the locations in the biopsies from the human patients. These particular cultures were not maintained over time because the embryos were harvested for histological examination. MJA has since been grown in culture using Vitacell RPMI-1640 culture medium with and without 20% fetal calf serum (ATCC) and in the allantoic fluid and other locations of fertilized chicken eggs.

Thus, MJA was discovered, and isolated and substantially purified, from its original tissue.

Some further characterization of MJA.

FIG. 1 is an electron micrograph of an MJA in an RBC.

FIGS. 2 & 3 show 1,000× blood smears with MJA in the RBCs and plasma. The blood smears were prepared as wet mounts of 1-2% Crystal Violet.

FIG. 4 shows a 1,000× histologic section of a Colon biopsy that has been Genta stained. The small arrow shows black aggregates of MJA within lining cells of the colon. The large arrow shows MJA scattered in the stroma. Nuclei of lining cells are not well seen, but the rows of lining cells can be identified.

FIG. 5 shows a 400× hemotoxylin and eosin (H&E) stained histologic colon section from a human with non-specific colitis. There is inflammation in the supporting stroma. MJA stained blue. Many of the surface cells are infected, with the MJA in substantially the same stage of development.

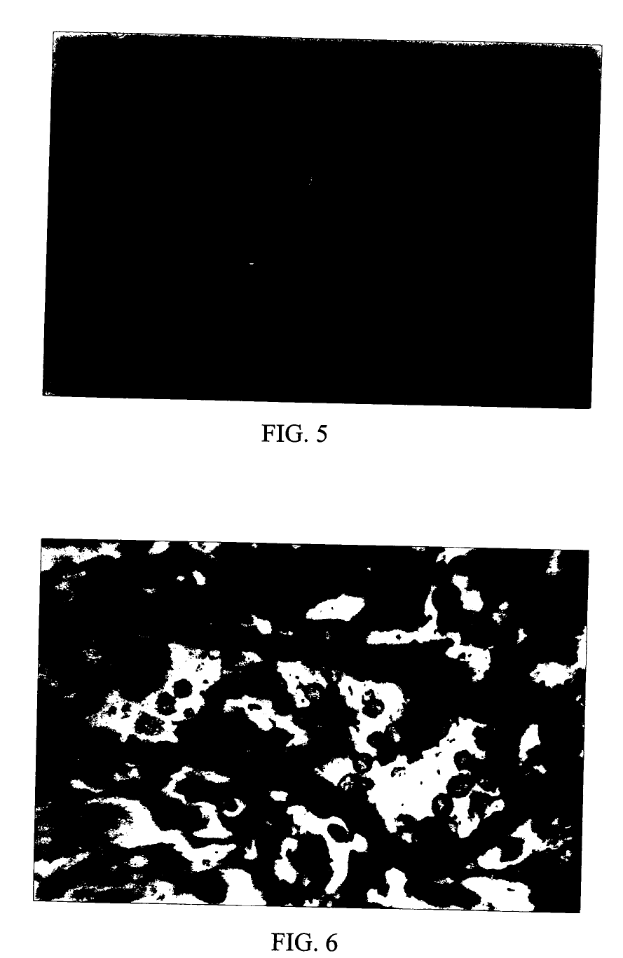

FIG. 6 shows a 1,000× Steiner stained small vessel in the colon. The vessel wall contains MJA and the lumen contains RBCs with MJA. MJA is also seen in plasma.

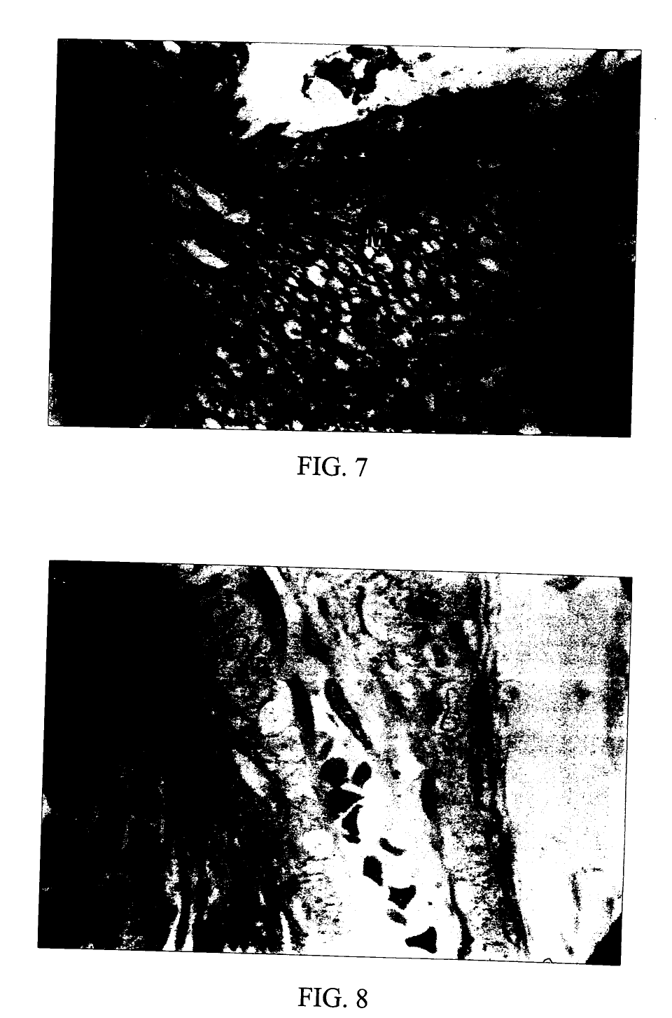

FIG. 7 shows a 1,000× medium sized blood vessel in the colonic muscle with MJA showing as black dots in the muscle.

FIG. 8 shows a 1,000× light photomicrograph thick section of the colon wall embedded in plastic and stained with toluidine blue for E. M. study. The black dots are MJA and can be seen intracellularly and intravascularly.

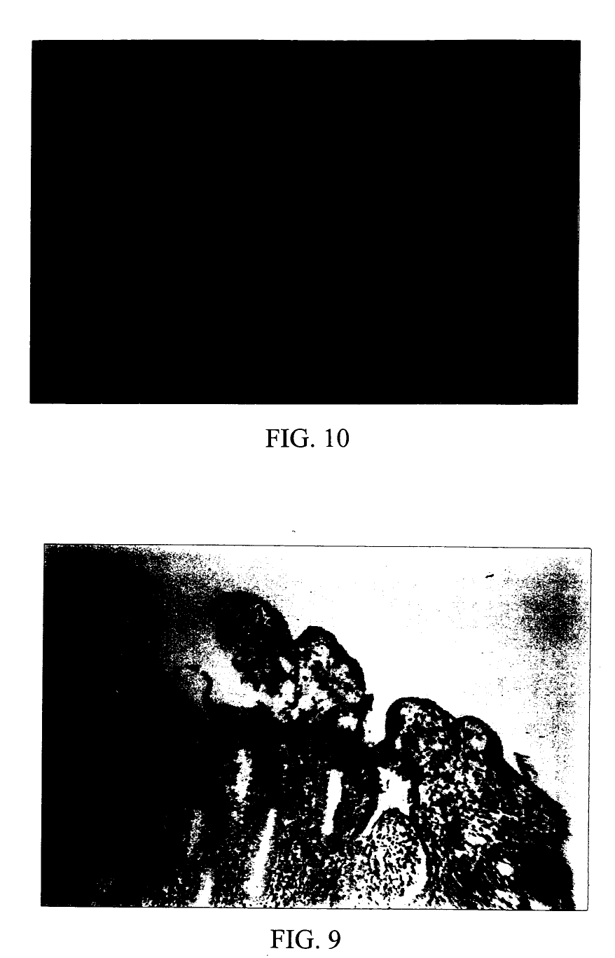

FIG. 9 shows a 400× histologic section of duodenum from a human with colitis. MJA stains black (arrows) and can be seen in small vessels, which are dilated and some are occluded. This pattern of vessels involvement produces swelling, redness and ulceration of the mucosa, which is similar to the pathogenesis of colitis.

FIG. 10 shows a 1,000× duodenum section from a human with abdominal discomfort, fatigue and a sick feeling The MJA are black and scattered through the stroma, in lymphatics and capillaries. The MJA vary in shape and size.

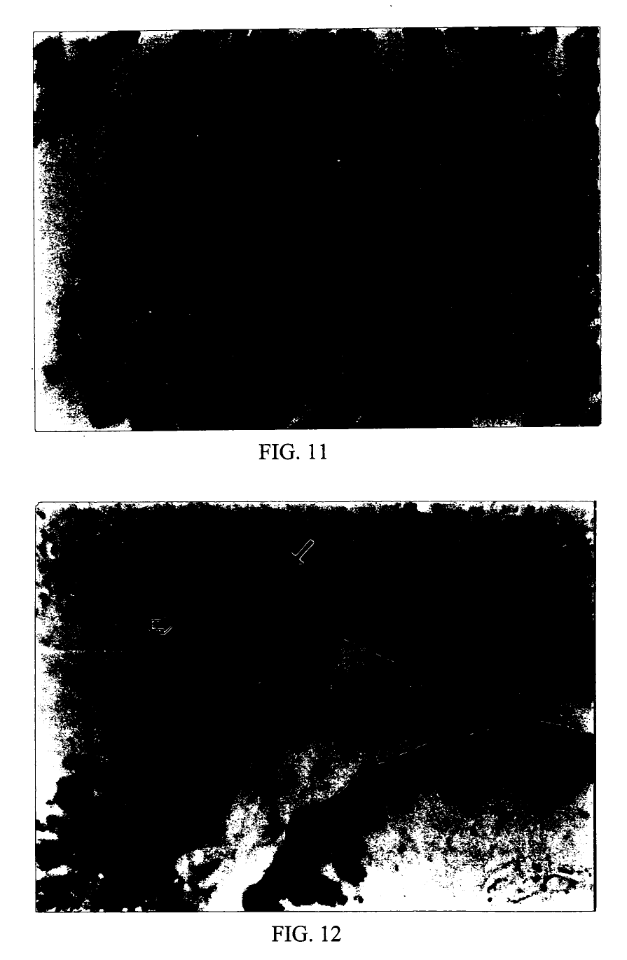

FIG. 11 shows a 1,000× photomicrograph of a Steiner stained blood vessel of a colon polyp removed by polypectomy. MJA appears as black dots in and on the RBCs in the lumen of the vessel. Larger dots appear to represent groups or clumps of MJA.

FIG. 12 shows a 1,000× Genta stained section of an ademnomatous polyp of the colon. MJA stained black and mucus stained blue. The closed arrow shows a cell full of MJA and the open arrow shows a cell with mucus stained blue and MJA stained black.

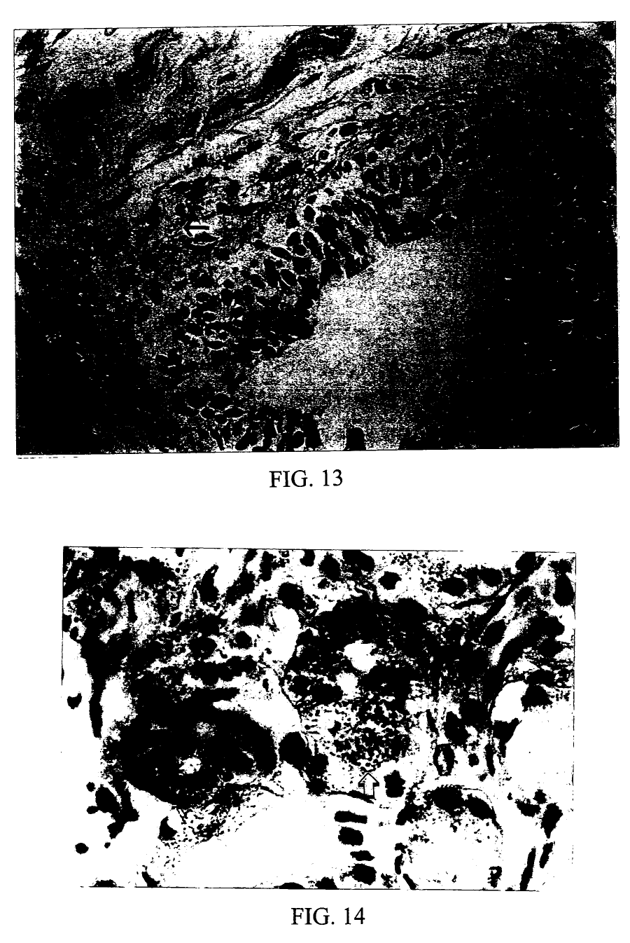

FIG. 13 shows 1000× Steiner stained tissue section from male breast from a human with gynecomastia. The veins and lymphatics around the ducts from male breast (arrow) show MJA, which appear as black dots.

FIG. 14 shows 1000× H&E stained section of salivary gland showing changes in lining cells and round forms of MJA.

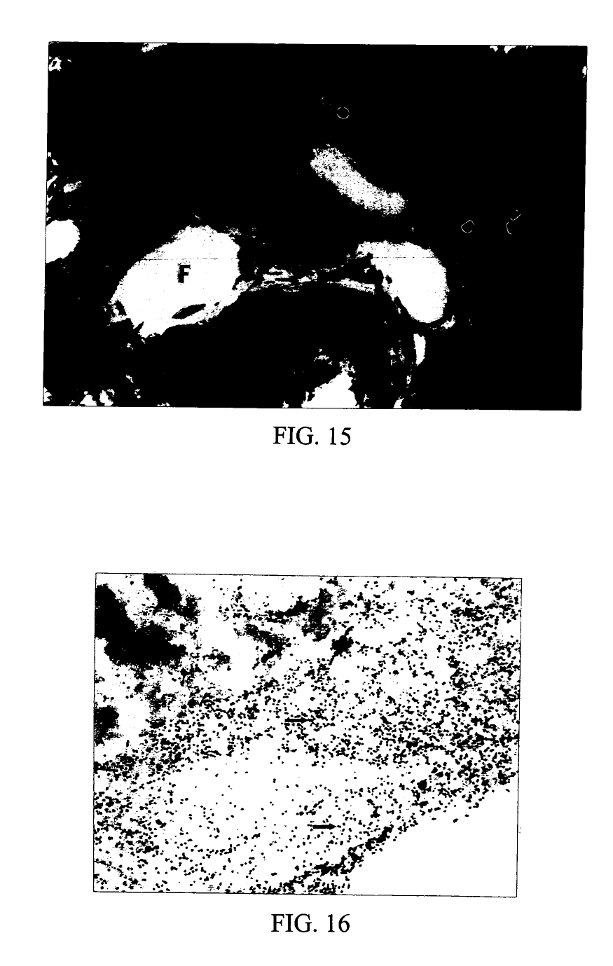

FIG. 15 shows 1000× H&E stained choroid plexus from a human that died at age 37 from diabetes mellitus. The picture was taken under UV light and through a filter that eliminated the excitation light. The lumens of the vessels are occluded by a fluorescent material (F) that appears to be related to MJA.

FIG. 16 shows 1000× Steiner stained histologic section of yolk sac from a fertilized chicken egg that had been infected with MJA. The MJA appear black and vary in size. The arrows point to individual MJA.

Based upon the information in the Figures and other observations, it appears that MJA has a complex life cycle and growth cycle. It can range in size from very small to large, for example from about 0.5 to 2.5 microns, and can aggregate to create a mass in the cell. It can leave cells. MJA can be found in a variety of cells and tissues as well as in interstitial spaces.

In the inventor's hands, MJA did not stain with Wright's stain, but did stain with Giemsa, the Steiner stain and crystal violet stain (MJA stained with crystal violet, but did not retain the stain when subjected to washing in the Gram stain protocol, thus MJA did stain with crystal violet but is also Gram negative). See, e.g., FIGS. 2-16. MJA found in red blood cells (RBCs) did not stain with hematoxylin although MJA showed up as blue dots when stained with hematoxylin in other locations. The reason for this failure to stain with hematoxylin is unknown, but MJA may be retained in vacuoles or other structures in the RBCs that prevent the stain from reaching MJA.

In order to determine whether MJA is potentially antigenic, colon tissue sections from four colitis patients infected with MJA were incubated with serum from the patient's own blood. If the tissue section contained an antigenic, and therefore likely pathogenic, microorganism the antibodies would attach to the microorganism. An immunohistochemical (IHC) test found antibodies bound to the tissue sections at the same sites where MJA was found by silver staining methods, which thus indicates that MJA is antigenic.

MJA has been found in the blood stream, and accumulated in the small vessels. Examples of this can be seen in most of the Figures, as discussed below and elsewhere herein. The levels of carbon dioxide and carbonic acid are high in the small veins, which may provide a positive environment for MJA.

Evidence of the potential pathogenicity of MJA includes the observation that the presence of MJA correlates with vascular blockage. MJA is also found in large numbers in the peritoneal cavity in peritonitis cases. MJA occurs in exudates of inflammatory cells, as do other pathogens. Also, as noted above, MJA is antigenic, which is often indicative of pathogenicity.

Some of the characteristics associated with MJA are consistent with features of various inflammatory and/or diarrheal disorders of the gastrointestinal (GI) tract, such as colitis, inflammatory bowel disease and many gastrointestinal cases with minimal symptoms. For example, early ulcerative colitis features redness of the mucosa with swelling of the mucosal folds and edema of the submucosa. As the disease advances, small hemorrhages and ulcers develop, then the size, wall shapes and motility of the intestine change. These symptoms are typical of small vessel obstruction, which as noted above is a characteristic of MJA. Lesions may be minor if the area is small and small numbers of MJA are involved.

In at least some cases of infection of the gastrointestinal tract, and particularly the small bowel (jejunum and ileum) some absorptive surface cells infected with MJA appear to change to resemble goblet cells. Most of the cells in the mucosal lining of the small bowel are normally absorptive cells; the inventor has observed in some cases over half infected by MJA. MJA can start off very small, then grow larger and can be eventually shed into the bowel lumen. Infected cells begin small, fill up with MJA then extrude the MJA into the bowel lumen.

When multiple biopsies were taken along an infected gastrointestinal tract, each biopsy shows MJA at substantially the same stage of development. FIG. 5 shows an example of such a biopsy, where multiple lining cells contain MJA in the same stage of development. It appears that MJA can divide and grow in synchrony, and such growth can be localized, even to an organ or a part of an organ. This growth pattern accords with the progression of explosive attacks in colitis. For example, a patient can appear to be well then one hour later be prostrate, sweating and immobilized, with diarrhea at one end and vomiting at the other. Bleeding, hemorrhage and shock may result in a surgical emergency or even death. Long-term consequences can include involvement of other organs, aging and development of cancer. During these acute attacks MJA has been found in large numbers in blockages of blood vessels.

The above comments and other perceptions of the inventor provide the following observations about MJA:

a. MJA may persist throughout the life of an individual, apparently causing periodic exacerbations of disease. Generally increasing damage is seen with increasing age. Intial infection can occur early in life and may possibly be present at birth.

b. Evidence of MJA is found in a large percentage of sick patients.

c. MJA is pleomorphic and has been found in spherical, elongated, pointed, and occasionally C-shaped forms. Various shapes of MJA have also been found when MJA has been grown in culture.

d. MJA's size is typically about 0.5 to 2.5 microns.

e. MJA has been seen with the following stains, a) Steiner stain, b) crystal violet stain, c) Genta stain, d) Gomori silver methenamine stain counter stained with hematoxylin, e) hematoxylin except inside RBCs, and f) Giemsa stain except inside RBCs. MJA has not been seen with the following stains, a) Wright stain as normally used in RBCs, b) Gram stain, c) eosin except as noted elsewhere herein, and d) acid fast stains.

f. MJA has been seen in electron micrographs. (See, e.g., FIG. 1.)

g. MJA has been grown in culture in Vitacell from ATCC, RPMI-1640 Medium, modified, ATCC Number: 30-2001, with and without 20% fetal calf serum, and in chick embryo cells, and in the allantoic fluid and other portions of fertilized hen's eggs.

h. MJA has been found blocking small blood vessels, which blockage is fluorescent when stained by Eosin Y, appears to produce ischemic changes with hemorrhage and ulcerations of small and large bowel, and conforms to the pathogenesis of ulcerative colitis and Crohn's disease.

i. MJA in some cases occurs in very large numbers with little or no inflammation, although in other cases it is sometimes found in association with inflammatory lesions.

j. In Crohn's disease and other inflammatory bowel disease (IBD), MJA appears to change absorptive cells of the small bowel into cells resembling goblet cells.

The sac-like structures in the cells, which resemble the sacs that are characteristic of real goblet cells, appear similar to parasitophorous vacuoles and contain MJA.

k. MJA binds to antibodies in a patient's serum when their own cells are incubated with the serum. Thus, MJA appears to be antigenic.

Turning now to some particular embodiments, the present invention comprises purified MJA (as exemplified by the deposit ATCC Accession No ______; MJA can be obtained from such deposit or from suitable natural sources, such as humans or other animals harboring MJA) as well as isolated and specifically identified MJA. The present invention also provides kits comprising a vessel containing MJA and a label specifically identifying the MJA. The vessel can be a vial, microscope slide or other suitable container. The MJA can be in an acellular culture medium, a cell culture medium, a tissue sample, or other suitable growth media. The kits can comprise one or more images of specifically identified MJA.

The present invention also comprises methods of obtaining MJA comprising: a) identifying an animal suspected of being infected with MJA; and, b) withdrawing a sample of tissue from the animal wherein the sample contains MJA. Similar methods can also serve to seek MJA in such an animal, for example a human patient, even where the sample does not include MJA. The methods can further comprise specifically identifying MJA from the tissue and, if desired, substantially purifying the MJA from the tissue and growing the MJA.

The present invention further comprises methods of specifically identifying MJA comprising: a) providing a sample of tissue suspected of containing MJA; b) examining the sample to determine the presence of MJA; and, c) specifically identifying MJA. The methods can further comprise, prior to the identifying, staining the sample with a stain suitable for MJA, and the examining can comprise magnifying the sample; other methods of detection, including indirect methods, can also be used. Where the methods comprise magnifying, the methods can also comprise creating a photomicrograph or other image of the MJA. The magnifying can be, for example, visible light microscopy, fluorescent light microscopy or electron microscopy (in which case the photomicrograph comprises an electronphotomicrograph), confocal microscopy or any other desired imaging modality. The image can be photographic, digital, videographic or otherwise as desired.

The present invention also provides kits comprising an image (hard copy or digital or other desired substrate) such as a photomicrograph and a label wherein the photomicrograph depicts at least one MJA and the label specifically identifies the MJA. The photomicrograph can be photographic paper and the label can be attached (e.g., glued or otherwise adhered) to the photomicrograph, the photomicrograph and the label can be digital representations that are digitally associated with each other, or combinations of the two or other imaging materials as desired. The photomicrograph can be a visible light photomicrograph, a fluorescent light photomicrograph, an electronphotomicrograph or other micrograph or imaging representation as desired.

### EXAMPLES

### Example 1

### Isolating, Culturing and Storing MJA From Blood.

Equipment and Media:

Incubator equipped with CO2 control, set at 5% CO2.

Cylinder of CO2.

Meter for CO2 cylinder, set at 15 PSI.

Vitacell RPMI-1640 culture medium with 20% fetal calf serum (ATCC).

Crystal Violet 2% aqueous.

Sterile pipettes, 2.0 ml and 10 ml.

Sterile centrifuge tubes, 10 ml or 2 ml.

Automatic pipettor.

Liquid nitrogen or −70 freezer.

Glass slides and cover slips.

Procedure:

Blood was collected from a colitis patient using buffered sodium citrate anticoagulant, and anticoagulated by gently swirling the mixture. 2 ml Vitacell were dispensed into sterile centrifuge tubes. 1 ml of whole blood was added to the tubes then mixed by gentle rotation. The mixtures were incubated at 39° C. in atmosphere of 5% CO2. The culture was checked at 24 and 48 hours. The culture turned color from pink to yellow tint as an indicator of cell growth.

To assay for the presence of MJA, 1 drop of culture (cells and fluid) was placed on a glass slide. 1-2 drops of aqueous crystal violet were added. A cover slip was wet mounted and the edges sealed with permount or fingernail polish then examined under a microscope. Erythrocytes from the blood contained MJA, which appeared as intracellular particles that stained with the crystal violet. MJA was also found in the supernatant fluid. Exemplary photomicrographs of blood/MJA prepared as discussed in this Example can be seen in FIGS. 2 & 3.

To cryopreserve, 10 ml of culture (2:1 Vitacell:blood) was mixed with 10 ml of a 7.5% glycerol in Ringers lactate solution. Cooling was carried out in a refrigerator. Aliquots (about 2 ml) were extracted and placed in 2 ml cryovials then placed in liquid nitrogen.

Two aliquots were retrieved from the liquid nitrogen, and warming was carried out slowly. The thawed samples were placed into further Vitacell medium and grown for 3 days at 37° C. Microscopic review found that the numbers of MJA in the 3-day old cultures had increased relative to the mixture after thawing.

The MJA from culture can also be used otherwise as desired.

Example 2

### Crystal Violet Staining.

Some samples of MJA were stained with crystal violet according to normal procedures except that the sample was stained fresh, unfixed as a wet mount. Crystal violet was applied to samples prepared as discussed in Example 2 as an aqueous solution at 1% or 2% w/v. The wet mount was sealed with finger nail polish. Microscopic examination was carried out promptly, before the sample could significantly degrade. Exemplary photomicrographs of blood/MJA prepared as discussed in this Example can be seen in FIGS. 2 & 3.

Example 3

### Growing MJA In Chicken Eggs

MJA was cultured in fertilized hens eggs, obtained disease free from Oregon State University Poultry Research (Corvallis, Oreg.), as follows.

Specimens:

The sources of MJA were:

Specimen A: Anticoagulated human whole blood containing the microorganism as seen in wet mounts stained with 1% crystal violet in physiologic saline. The blood had been collected from a patient with diarrhea and bronchitis and had been stored in liquid nitrogen for three weeks.

Specimen B: Two 2 ml cryovials prepared as set forth in Example 1 each containing pooled human blood samples that showed MJA on wet mount. The cryovials were stored in liquid nitrogen. For use, the cryovials were removed from the liquid nitrogen and thawed at room temperature (70° F.) for several hours.

Inoculation and Growth:

Six chicken eggs 6 days post-fertilization were used for inoculation, which was performed at room temperature. 6 additional eggs served as controls. The eggs were candled to locate the embryo and to confirm viability. The air sac was found at the blunt end. This end was sterilized with iodine and wiped with propyl alcohol. A Dremel® rotary tool was used to make a small hole in this end, over the air sac. The area was swabbed again with iodine and allowed to dry. 0.5 ml of each of the source specimens were aspirated through a 22 gauge, 1 inch needle into a 3 ml syringe. The needle was inserted along the long axis of the egg through the hole, aimed in front of the embryo. Slow injection of the 0.5 ml inoculant was performed and the needle withdrawn. The inoculation site was wiped with iodine, allowed to dry and sealed with epoxy glue.

Specimen A was inoculated into two eggs, Nos. 1 and 2.

Each cryovial of Specimen B was inoculated into two eggs, Nos. 3 and 4, and Nos. 5 and 6, respectively.

The eggs were incubated at 97° F. for 7 days in an incubator with a water dish. The eggs were rotated frequently, end over end, to inhibit adhesions of the embryo.

Harvesting:

The eggs were refrigerated at 34° F. for 18 hours prior to harvesting. This shrank the blood vessels and reduced blood contamination of fluid chambers. The eggs were washed in propyl alcohol. The area over the air sac was wiped with iodine. The shell was cracked with a blunt object and pieces of shell removed, revealing the shell membrane. An area 1 inch square was uncovered. The shell membrane was pierced and removed revealing the allantoic cavity. The allantoic fluid was greatly increased relative to the control eggs.

This allantoic fluid was aspirated into a 10 ml syringe and then placed in a 15 ml sterile centrifuge tube. 7-8 ml of allantoic fluid were aspirated from eggs Nos. 1 and 2 (from Specimen A; not all the fluid was removed), 7-8 ml of allantoic fluid were aspirated from eggs Nos. 3 and 4 (from Specimen B; not all the fluid was removed), while about 6-8 ml allantoic fluid was removed from each of eggs Nos. 5 and 6 (Specimen B).

The allantoic fluid was cloudy gray. A drop was placed on a microscope slide. One or two drops of crystal violet solution were added. The preparation was cover slipped and sealed with nail polish. Examination under the microscope showed about a 4 times increased density of MJA compared to the specimens prior to inoculation. Since about 0.5 ml of inoculum was injected and 15 ml of allantoic fluid were recovered, it is estimated that the total number of MJA increased about 120 times.

Allantoic fluid from 3 eggs was placed in 30 numbered, sterile cryovials as follows:

Egg No. 2 (Specimen A): 0.5 ml in cryovials 1-8.

Egg No. 6 (Specimen B): 0.5 ml placed in cryovials 9-20.

Egg No. 5 (Specimen B): 0.5 ml placed in cryovials 21-30.

The cryovials were refrigerated 2 hours at 34° F. and plunged in liquid nitrogen.

15 ml test tubes containing allantoic fluid from eggs Nos. 4, 5 and 6 were also placed straight into liquid nitrogen. Four cryovials each containing 0.5 ml allantoic fluid from egg No. 4 were fixed in 1.5 ml glutaraldehyde for electron microscopy.

Three cryovials containing pieces of yolk sac were also placed in liquid nitrogen. The remaining content of the egg (the yolk) was decanted into a sterile Petri dish after first separating the yolk sac from the embryo. The yolk sac was isolated in the Petri dish. Its membrane was incised and the membrane removed and fixed in 10% formalin.

The remainder of the egg was discarded.

### Example 4

### Histology on Yolk Sacs From Example 4.

Yolk sacs were isolated from every egg from Example 4 to provide 6 infected samples and 6 control samples. The specimens were embedded in paraffin and sections from each were H&E stained and Steiner stained. The Steiner stain on the 6 infected eggs showed MJA in large numbers. The infected eggs showed MJA in blood, in RBCs, in blood vessels, in yolk sac cells and in the yolk. A photomicrograph of a yolk sac prepared as discussed in this Example can be seen as FIG. 16. The H&E stains were inconclusive as to the presence of MJA but were helpful in identifying structures in the specimens. Steiner stain on the 6 control eggs showed no MJA.

### Example 5

### Re-Growing MJA In Chicken Eggs.

Two cryovials containing pure allantoic fluid infected with MJA from Example 3 were thawed at room temperature then used to infect new fertilized chicken eggs according to the procedure set forth in Example 3. The new chicken eggs grew MJA, as seen by microscopic examination of the allantoic fluid.

### Example 6

### Determination of Antigenicity of MJA.

To determine if four colitis patients infected with MJA (as determined by silver staining) carried antibodies against MJA, tissue sections of the colon from the four patients were incubated with the patient's own serum under routine conditions. Mouse anti-human gamma globulin antibodies were applied to the tissue sections under routine conditions. The presence of binding was determined using the immuno-peroxidase method. Binding of the mouse antibodies was observed at the same sites where MJA was found by silver staining sections of the same biopsies. This indicates that MJA is antigenic.

### Example 7

Fresh human blood from a colitis patient was smeared on a slide and a wet-mount stained with 2% crystal violet. The sample contained large numbers of MJA. FIG. 2. Further fresh blood from the patient was spread with a sterile loop on culture plates containing media. Two media (PML Microbiologicals, Willsonville, Oreg.) were used: 1) TSA with 5% sheep blood, or 2) Chocolate agar with enrichment. The plates incubated for 12 days at 37° C. There was no growth on the culture plates.

This indicates that MJA does not grow on these two commonly used solid culture media.

From the foregoing, it will be appreciated that, although specific embodiments have been discussed herein for purposes of illustration, various modifications may be made without deviating from the spirit and scope of the disclosure. Accordingly, the systems and methods, etc., include such modifications as well as all permutations and combinations of the subject matter set forth herein and is not limited except as by the appended claims.

What is claimed is:

1. Substantially purified microorganism John Antonius (ATCC Accession No. ______).

2. Isolated and specifically identified microorganism John Antonius (ATCC Accession No. ______).

3. A kit comprising a vessel containing isolated microorganism John Antonius (ATCC Accession No. ______) and a label specifically identifying the microorganism John Antonius.

4. A kit comprising a vessel containing substantially purified microorganism John Antonius (ATCC Accession No. ______) and a label specifically identifying the microorganism John Antonius.

5. The kit of claim 3 or 4 wherein the vessel is a vial.

6. The kit of claim 3 or 4 wherein the vessel is a microscope slide.

7. The kit of claim 3 or 4 wherein the microorganism John Antonius is in an acellular culture medium.

8. The kit of claim 3 or 4 wherein the microorganism John Antonius is in a cell culture medium.

9. The kit of claim 3 wherein the microorganism John Antonius is in a tissue sample.

10. A method of obtaining microorganism John Antonius comprising:
a) identifying an animal suspected of being infected with microorganism John Antonius (ATCC Accession No. ______); and,
b) withdrawing a sample of tissue from the animal wherein the sample contains microorganism John Antonius.

11. The method of claim 10 wherein the method further comprises specifically identifying microorganism John Antonius from the tissue.

12. The method of claim 11 wherein the method further comprises substantially purifying the microorganism John Antonius from the tissue.

13. The method of claim 12 wherein the method further comprises growing the microorganism John Antonius in a culture medium.

14. A method of specifically identifying microorganism John Antonius comprising:
a) providing a sample of tissue suspected of containing microorganism John Antonius (ATCC Accession No. ______);
b) examining the sample to determine the presence of microorganism John Antonius; and,
c) specifically identifying microorganism John Antonius.

15. The method of claim 14 wherein the method further comprises, prior to the identifying, staining the sample with a stain suitable for microorganism John Antonius.

16. The method of claim 14 or 15 wherein the examining comprises magnifying and the method further comprises, after the magnifying, creating an image of the microorganism John Antonius.

17. The method of claim 16 wherein the magnifying comprises visible light microscopy.

18. The method of claim 16 wherein the magnifying comprises fluorescent light microscopy.

19. The method of claim 16 wherein the magnifying comprises electron microscopy and the image comprises a photomicrograph.

20. The method of claim 19 wherein the photomicrograph comprises an electronphotomicrograph.

21. The method of claim 19 wherein the photomicrograph comprises a visible light photomicrograph.

22. The method of claim 19 wherein the photomicrograph comprises a fluorescence photomicrograph.
